(19) United States
(12) Patent Application Publication
(10) Pub. No.: US 2020/0085169 A1
(43) Pub. Date: Mar. 19, 2020

(54) MAGNETIC COSMETIC APPLICATOR

(71) Applicant: HCT GROUP HOLDINGS LIMITED, SHEUNG WAN (CN)

(72) Inventors: DENIS PIERRE MAURIN, LOS ANGELES, CA (US); JAE BIN, LOS ANGELES, CA (US); THOMAS SANG CHUN, BUENA PARK, CA (US)

(73) Assignee: HCT GROUP HOLDINGS LIMITED, SHEUNG WAN (CN)

(21) Appl. No.: 16/571,926

(22) Filed: Sep. 16, 2019

Related U.S. Application Data
(60) Provisional application No. 62/732,302, filed on Sep. 17, 2018.

(51) Int. Cl. A45D 40/26; A61K 8/19; A61Q 1/02

(52) U.S. Cl. CPC A45D 40/267; A45D 40/264; A45D 2200/1009; A61Q 1/02; A61K 8/19

(57) ABSTRACT

A cosmetic container and applicator system for magnetic cosmetic products may include a housing defining a reservoir for containing a magnetic cosmetic product, and an applicator configured to extend into the reservoir. The applicator includes a magnet. A wiper may extend into the reservoir and have a bottom end with an opening through which the applicator extends. The wiper may be configured to remove excess product from the applicator as the applicator is withdrawn from the reservoir.

### CROSS REFERENCE TO RELATED APPLICATIONS

The present application claims the benefit of and priority to U.S. Provisional Application Ser. No. 62/732,302, filed on Sep. 17, 2018, titled MAGNETIC COSMETIC APPLICATOR, the disclosure of which is incorporated herein by reference.

### BACKGROUND

Vessels exist that are convenient to use, and designed to contain and dispense cosmetic products for use. Cosmetic containers often include an applicator for removing a portion of the cosmetic product from the container. New and alternative designs are desired.

### Overview

This disclosure provides design, material, manufacturing methods, and use alternatives for cosmetic packaging.

According to one example of the present disclosure, a cosmetic applicator for magnetic cosmetic products comprises a housing defining a reservoir for containing a magnetic cosmetic product, an applicator configured to extend into the reservoir, the applicator including a magnet, and a wiper extending into the reservoir, the wiper having a bottom end with an opening through which the applicator extends, the wiper configured to remove excess product from the applicator as the applicator is withdrawn from the reservoir.

Alternatively, or additionally, in another example, the applicator includes a cap and an elongate stem, at least a portion of the elongate stem configured to extend through the opening in the wiper and into the reservoir when the cap is attached to the housing.

Alternatively, or additionally, in another example, the magnet is completely enclosed within the elongate stem.

Alternatively, or additionally, in another example, a majority of the stem extends through the opening in the wiper and into the reservoir when the applicator is attached to the housing.

Alternatively, or additionally, in another example, a majority of the stem is disposed within the wiper above the opening when the applicator is attached to the housing.

Alternatively, or additionally, in another example, the magnet is disposed within a recess in an end of the elongate stem.

Alternatively, or additionally, in another example, at least a portion of the magnet is in direct contact with cosmetic product when the stem is disposed within the reservoir.

Alternatively, or additionally, in another example, a majority of the stem extends through the opening in the wiper and into the reservoir when the applicator is attached to the housing.

Alternatively, or additionally, in another example, an entirety of the magnet is disposed within the reservoir when the applicator is attached to the housing.

Alternatively, or additionally, in another example, a majority of the stem is disposed within the wiper above the opening when the applicator is attached to the housing.

Alternatively, or additionally, in another example, the magnet is disposed with the recess such that only a single end surface of the magnet is exposed and in direct contact with cosmetic product when the stem is disposed within the reservoir.

Alternatively, or additionally, in another example, the magnet is an elongate magnet extending from the end of the elongate stem, wherein sides and an end surface of the magnet are exposed and in direct contact with cosmetic product when the stem is disposed within the reservoir.

Alternatively, or additionally, in another example, only the magnet extends through the opening in the wiper and into the reservoir when the applicator is connected to the housing.

Alternatively, or additionally, in another example, the cosmetic applicator further comprising a collar attached to an upper open end of the housing, the collar connecting the applicator to the housing.

Alternatively, or additionally, in another example, the bottom end of the wiper is disposed adjacent a bottom end of the reservoir.

Alternatively, or additionally, in another example, the housing has a neck region defining an open end of the reservoir, wherein the bottom end of the wiper is disposed with the neck region of the housing.

Alternatively, or additionally, in another example, the applicator takes the form of a mascara brush.

Alternatively, or additionally, in another example, the applicator takes the form of a sponge.

Alternatively, or additionally, in another example, the cosmetic applicator further comprises a magnetic cosmetic product contained in the reservoir.

In another example, a cosmetic applicator for magnetic cosmetic products comprises a housing, an applicator stem attached to the housing, the applicator stem including a magnet, and a cap removably connected to the applicator stem, the cap covering an entirety of the applicator stem.

Alternatively, or additionally, in another example, at least one surface of the magnet is exposed.

Alternatively, or additionally, in another example, an entirety of the magnet is enclosed within the applicator stem.

The above summary of some example embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of an illustrative cosmetic container;

FIG. 2 is an exploded perspective view of the illustrative cosmetic container of FIG. 1;

FIG. 3 is a cross-sectional view of the illustrative cosmetic container of FIG. 1, taken along line 3-3;

FIG. 4 is a cross-sectional view of the illustrative cosmetic container of FIG. 1 with the applicator separated from the housing;

FIGS. 5-9 are cross-sectional view of additional illustrative cosmetic containers;

FIG. 10 is a perspective view of an illustrative cosmetic applicator with a cap removed;

FIG. 11 is a cross-sectional view of the illustrative cosmetic applicator of FIG. 10, taken along line 11-11; and

FIG. 12 is a cross-sectional view of another illustrative cosmetic applicator.

### DETAILED DESCRIPTION

Cosmetic products with magnetic properties are becoming more popular. Products including skincare such as masks, hair products such as root touchup powder, and makeup such as mascara may contain magnetic particles. An applicator containing a magnet may improve the delivery and application of such magnetic products. In some cases, a magnetic applicator may allow for the delivery of a defined amount of product.

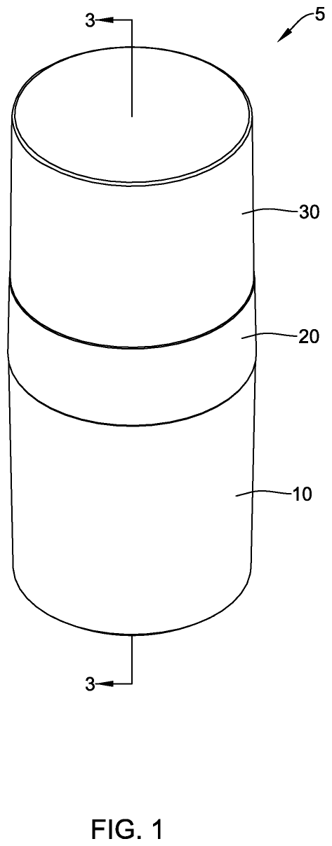

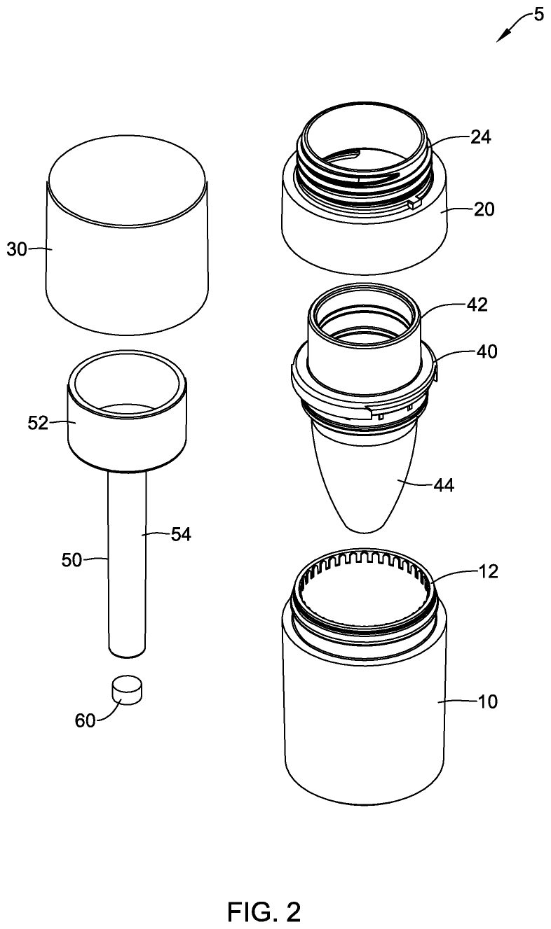

FIG. 1 shows an illustrative cosmetics applicator and container 5, including a housing 10, collar 20, and cap 30. The interior components of the cosmetics applicator and container 5 are shown in FIG. 2. The housing 10 may have a neck region 12 at the open upper end. The neck region 12 may have external threads, a groove or ridge to provide a snap fit, or may be sized to achieve a friction fit with the collar 20. An insert 40 may be disposed within the interior of the housing 10. The insert 40 may include an upper ring 42 configured to fit within the collar 20, and a tapered wiper 44 extending downward into the housing 10. The collar 20 may have an upper threaded neck 24 configured to mate with internal threading on the cap 30. An applicator 50 may include an upper portion 52 and an elongate stem 54. The upper portion 52 may be fixedly attached to the cap 30 such that removing the cap 30 from the collar 20 and housing 10 removes the applicator 50. In some embodiments, the cap 30 and applicator 50 may be a single monolithic piece. A magnet 60 may be attached to the end of the stem 54. The applicator stem 54 is shown as a straight rod, though other applicator shapes may be used, such as a mascara type brush, doe-foot applicator, or sponge. The magnet 60 may be disposed in the interior of such applicators.

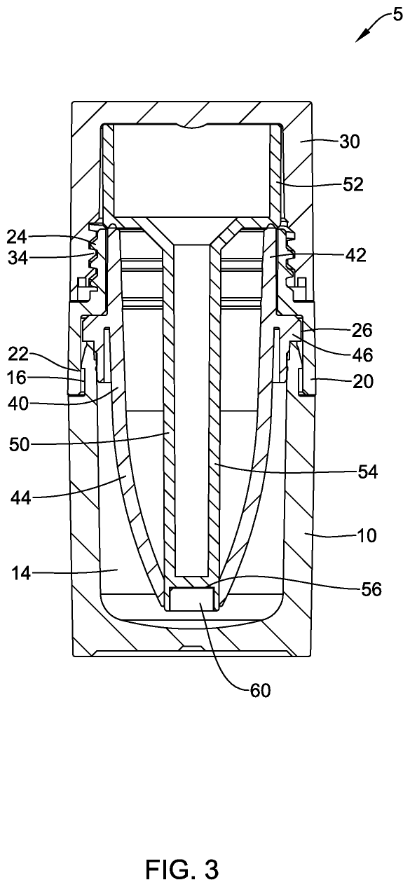

The cross-sectional view in FIG. 3 illustrates the interaction of the interior components of the cosmetics applicator and container 5. Interior threading 34 on the cap 30 mates with an exterior threaded neck 24 on collar 20. Other structures for securing the cap 30 to the collar 20 may be used instead. The collar 20 and housing 10 are non-rotatably connected. As illustrated, the collar 20 and housing 10 have a ridge and groove connection. The collar 20 has at least one ridge 22 sized and shaped to be received in a groove 16 extending around the neck region 12 of the housing 10. In other embodiments, groove may be on the collar and the ridge may be on the housing, or the collar 20 and housing 10 may be attached with a threaded connection or be connected with adhesive or via ultrasonic welding. The upper ring 42 of the insert 40 fits within the threaded neck 24 of the collar 20. The insert 40 may have a flange 46 that is received within an upper groove 26 on the collar 20. In the embodiment shown in FIG. 3, the insert 40 includes a wiper 44 extending into the reservoir 14 of the housing 10. The reservoir 14 contains the magnetic cosmetic product. The stem 54 of the applicator 50 may be an elongate member that extends through an opening in the bottom end of the wiper 44 such that at least the bottom of the stem 54 is in contact with the cosmetic product in the reservoir 14. In the embodiment shown in FIG. 3, the magnet 60 is received in the recess 56 at the bottom end of the stem 54, with only the bottom surface of the magnet 60 exposed to and in direct contact with the cosmetic product in the reservoir 14. The magnet 60 may be affixed within the recess 56 with an adhesive or by a friction fit.

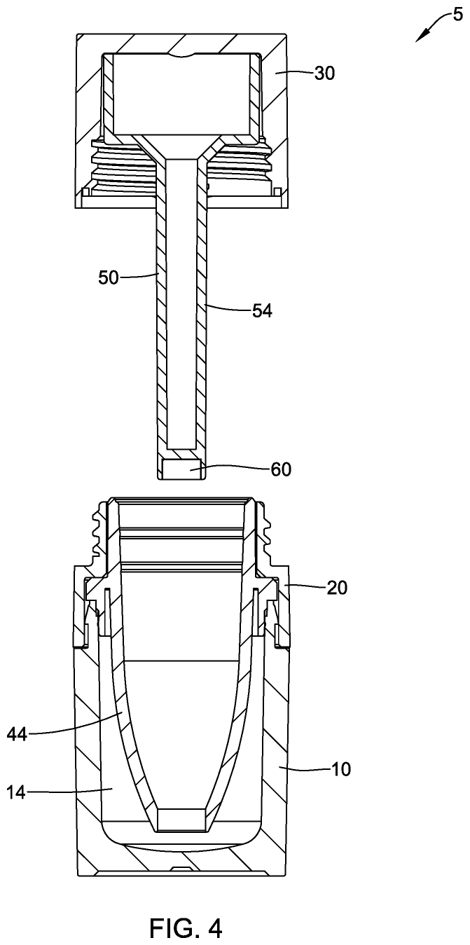

In use, the cap 30 is unscrewed from the collar 20 and removed, as shown in FIG. 4. A portion of cosmetic product will be magnetically adhered to the magnet 60 on the stem 54. The wiper 44 may help control the amount of product that is removed with the applicator 50, keeping the contact with the product limited to the bottom surface of the stem 54.

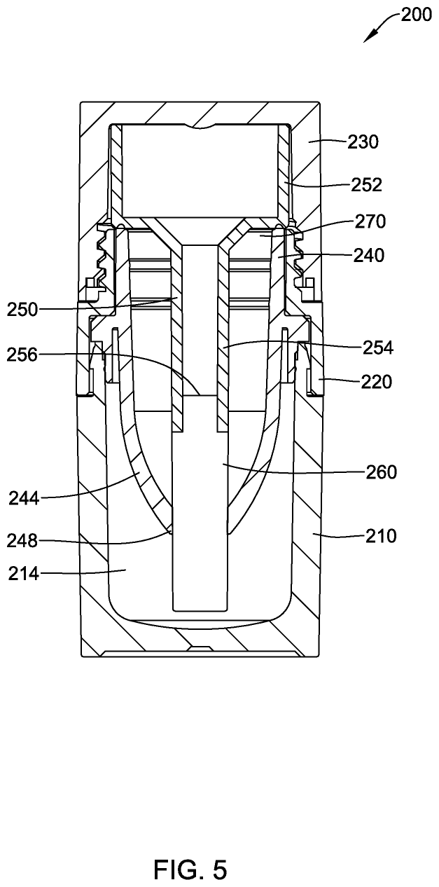

FIG. 5 shows a cross-sectional view of another illustrative cosmetic container and applicator 200 having a cap 230, collar 220, and housing 210 with similar structures and connections as the cap 30 collar 20, and housing 10 described above, with the addition of a liner 270. The liner 270 is disposed between the lower edge of the upper portion 252 of the applicator 250 and the top of the insert 240 and housing 210. The liner 270 may provide an airtight seal around the applicator stem 254. In the example shown in FIG. 5, wiper 244 does not extend as far into the reservoir 214 as in the example shown in FIG. 3, and the applicator 250 includes a shorter stem 254 and larger magnet 260. In this example, the stem 254 resides completely within the insert 240. The magnet 260 is received within a recess 256 in the bottom end of the stem 254, and only the magnet 260 extends through the opening in the wiper 244 and into the reservoir 214. The magnet 260 is shown as an elongate rod, although other shapes of magnet may be provided, such as oval, polygonal, etc. The elongate magnet 260 provides an increased surface area of contact with the product within the reservoir 214 when the cap 230 is attached to the collar 220. As the applicator 250 is removed from the housing 210, the wiper 244 will remove excess product from the sides of the magnet 260. The wiper 244 may include a flexible portion 248 that surrounds the opening and is in contact with the magnet 260. The flexible portion 248 may sweep excess product off the magnet 260 as the magnet 260 is withdrawn through the wiper 244.

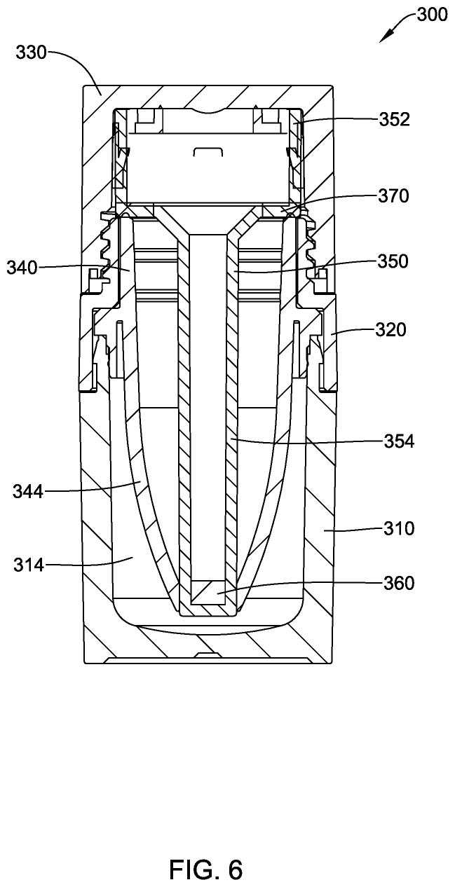

FIG. 6 shows a cross-sectional view of another illustrative cosmetic container and applicator 300 having a cap 330, collar 320, insert 340, and housing 310 with similar structures and connections as the cap 30 collar 20, insert 40, and housing 10 described above, with the addition of a liner 370. The liner 370 is disposed between the lower edge of the upper portion 352 of the applicator 350 and the top of the insert 340 and housing 310. The liner 370 may provide an airtight seal around the applicator stem 354. In the example shown in FIG. 6, the magnet 360 is completely enclosed within the elongate stem 354 of the applicator 350. The stem 354 may be made of plastic and may be rigid, soft, or flexible. The thickness of the stem 354 covering the magnet 360 may be selected so as to not interfere with the magnetic properties of the magnet 360. The wiper 344 extends well into the reservoir 314, similar to the wiper 44 shown in FIG. 3, such that only the bottom end of the stem 354 contacts the product.

The cosmetic container and applicator assemblies shown in FIGS. 1-6 all include a collar configured to connect the cap and housing. It will be understood that each assembly may also be formed without the collar, in which case, the housing would be connected directly to the cap and the insert would be connected directly to the housing.

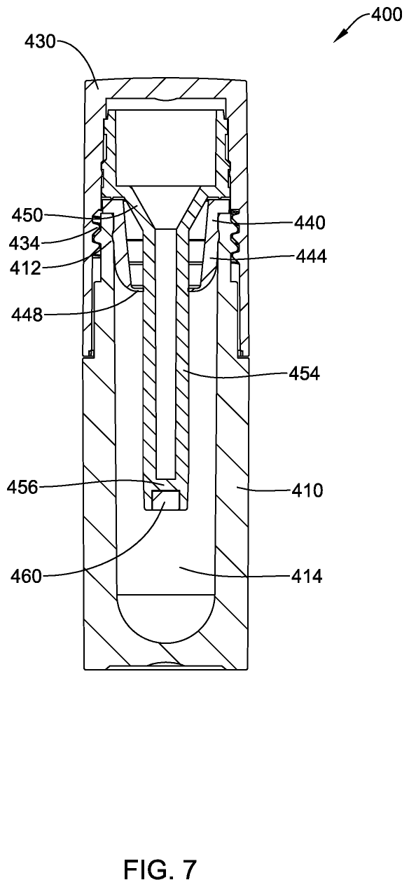

FIGS. 7-9 illustrate example cosmetic containers and applicators without a collar. As seen in FIG. 7, the cosmetic container and applicator 400 has a cap 430 with internal threading 434 that engages an externally threaded neck 412 on the upper end of the housing 410. The applicator 450 is fixedly attached to the inner surface of the cap 430. The insert 440 includes a wiper 444 that only extends into the neck 412 of the housing 410, and the stem 454 of the applicator 450 extends through the wiper 444 such that a majority of the stem 454 resides within the reservoir 414. The magnet 460 resides within a recess 456 at the bottom end of the stem 454. The reservoir 414 in this example cosmetic container and applicator 400 is larger than the reservoirs of the previous containers. While more of the stem 454 is in contact with the product in the reservoir 414, excess product will be removed as the stem 454 is withdrawn through the wiper 444. The entire wiper 444 may be flexible, or it may have a flexible region 448 adjacent the opening for receiving the stem 454.

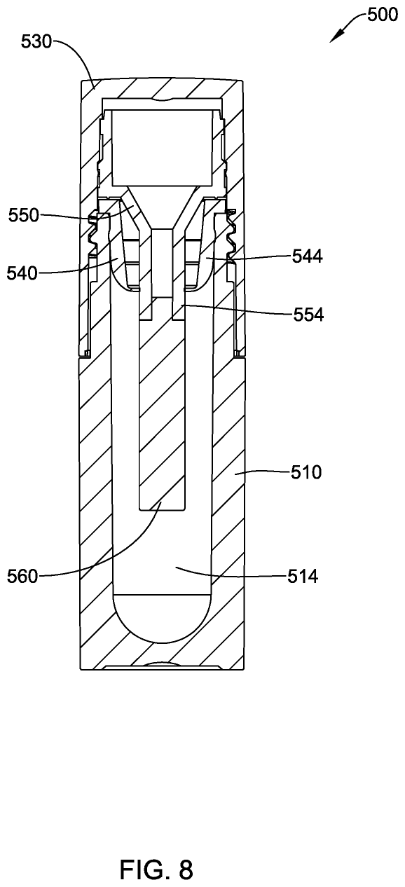

FIG. 8 shows a cross-sectional view of another illustrative cosmetic container and applicator 500 having a cap 530, insert 540, and housing 510 with similar structures and connections as the cap 430, insert 440, and housing 410 described above with regard to FIG. 7. The applicator 550 is fixedly attached to the inner surface of the cap 530, and the applicator 550 has a similar structure to the applicator 250 shown in FIG. 5, with a relatively short stem 554 and elongated magnet 560 attached to the bottom end of the stem 554. The entire magnet 560 resides with the reservoir 514 and is in direct contact with the product, however, as the applicator 550 is withdrawn from the reservoir 514, the wiper 544 removes excess product from the sides of the magnet 560.

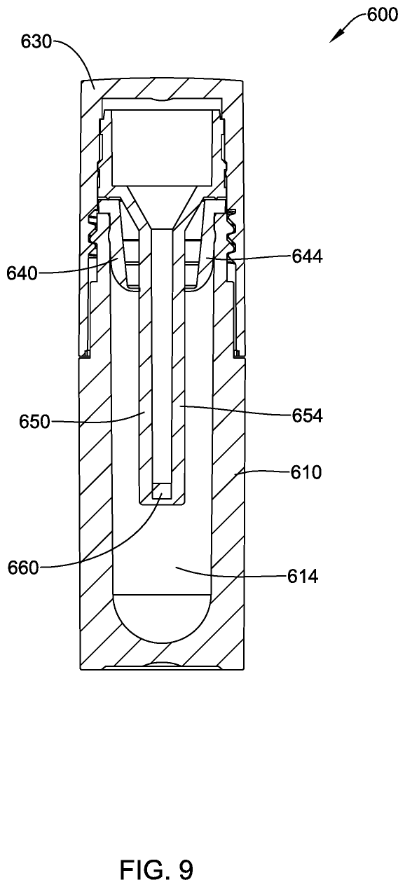

FIG. 9 shows a cross-sectional view of another illustrative cosmetic container and applicator 600 having a cap 630, insert 640, and housing 610 with similar structures and connections as the cap 430, insert 440, and housing 410 described above with regard to FIG. 7. The applicator 650 is fixedly attached to the inner surface of the cap 630, and the applicator 650 has a similar structure to the applicator 350 shown in FIG. 6, with the magnet 660 completely enclosed within the elongate stem 654 of the applicator 650. A majority of the stem 654 resides with the reservoir 614 and is in direct contact with the product, however, as the applicator 650 is withdrawn from the reservoir 614, the wiper 644 removes excess product from the sides of the stem 654.

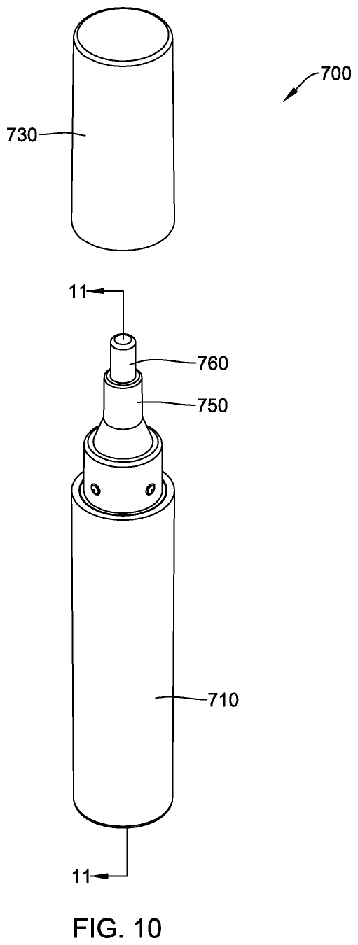

FIGS. 10-12 illustrate example cosmetic application devices 700, 800 that do not include a container or reservoir for holding a product. The application devices 700, 800 may be used to collect and apply a product having magnetic properties from a separate container. As seen in FIG. 10, the application device 700 may include a cap 730 and a housing 710 having an applicator 750 attached thereto. The applicator 750 may include a magnet 760. In some examples, the application device 700 may have a pen or stick shape, as shown in FIG. 10. In other examples, the application device may have any shape such as an oval, sphere, prism, or polygon.

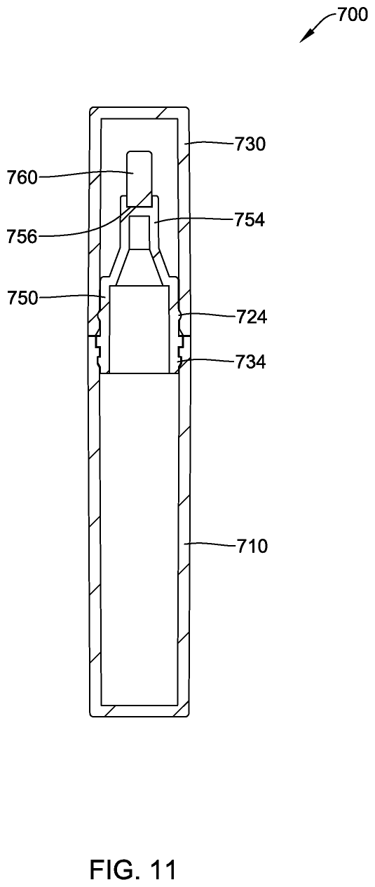

The internal components of the application device 700 are shown in the cross-sectional view of FIG. 11. The cap 730 may be connected to the applicator 750 via a threaded connection, friction fit, or snap fit such as with a series of projections 724 on the outer surface of the applicator 750 that frictionally engage the inner surface of the cap 730 or a recess on the inner surface of the cap 730. The housing 710 may engage the applicator 750 via a threaded connection, friction fit, or a snap fit such as via a ridge 734 on the lower portion of the applicator 750 engaging a recess on the inner surface of the housing 710. The applicator 750 includes a stem 754 extending upwards from the housing 710 and the stem 754 has a recess 756 configured to receive an end of the magnet 760. The magnet 760 may extend above the top surface of the stem 754, such that the magnet alone may be inserted into and directly contact the cosmetic product.

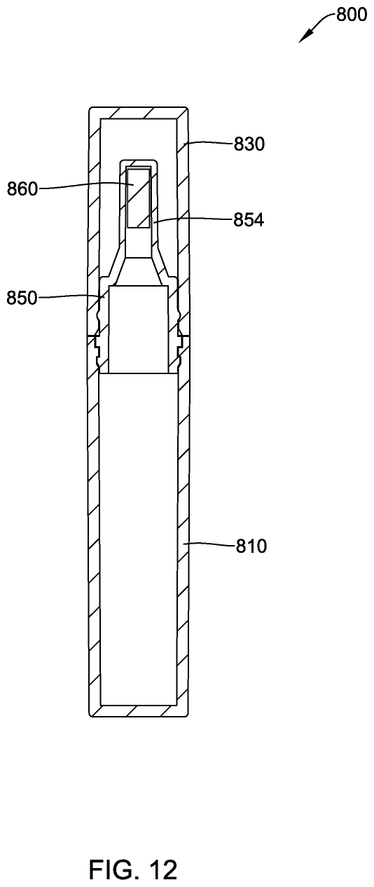

FIG. 12 shows a cross-sectional view of another illustrative application device 800 having a cap 830 and housing 810 with similar structures and connections as the cap 730 and housing 710 described above with regard to FIG. 11. The applicator 850 has a similar structure to the applicator 750 shown in FIG. 11, except the magnet 860 is completely enclosed within the stem 854 of the applicator 850.

The cosmetics applicator and container 5, 200, 300, 400, 500, 600, and the cosmetics application devices 700 and 800 may all be used with various forms of cosmetics products, such as fine to thick powders with and without a binder, viscous materials, gels, liquids, creams, and pastes. The relatively small magnets 60, 360, 460, 660 shown in FIGS. 3, 6, 7, and 9 may provide for the pickup and delivery of a defined amount of cosmetic product each time the applicator is inserted into the reservoir. In particular, the single magnetic contact surface provided with magnets 60, 460 having a single exposed lower surface, as shown in FIGS. 3 and 7, may provide a defined amount of product. The exposed surface area of the exposed magnet and the strength of the magnet may be varied to achieve a specific amount of product to be adhered to the applicator 50, 450 as it is withdrawn from the housing 10, 410. The magnets 60, 260, 360, 460, 560, 660, 760, 860 may be made of any conventional magnetic material, and may include any magnetic ferrous metal or alloy.

The cosmetics applicator and container 5, 200, 300, 400, 500, 600, and the cosmetics application devices 700 and 800 and their component parts as shown and described above may be made of any suitable material such as, for example and without limitation, thermoplastic elastomer (TPE), low density polyethylene (LDPE), synthetic polymer, partially of a resin such as, for example, acrylonitrile butadiene styrene (ABS), styrene acrylonitrile (SAN), hard plastic such as polymethyl methacrylate (PMMA), polycyclohexylenedimethylene terephthalates (PCT, PCTG, PCTA), polyethylene terephthlates (PET, PETE, PETG), polypropylene (PP), polyethylene (PE), polyurethane, rubber, silicone, ceramic, glass, metal, wood, or composite material, and/or combinations thereof. Moreover, various elements may be made of any combination of substantially clear, substantially opaque, and/or translucent materials.

While the illustrated examples show a rod shaped applicator, the present invention may also be used with a mascara brush, a sponge, a flocked applicator, a doefoot applicator, or any other applicator. The magnet 60 may be disposed within a mascara brush, sponge, flocked applicator, or doefoot. Mascara, face masks, and hair coloring powder are some magnetic cosmetic products that may be used with the described containers and applicators. In other examples, products such as lip gloss, face powder, concealer, eye shadow, and other products containing magnetic compounds may be used with the descried containers and applicators.

Each of the above non-limiting examples can stand on its own, or the various elements of each example can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the following claims, the terms “first,” “second,” and “third,” etc. are used merely as labels, and are not intended to impose numerical requirements on their objects. The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A cosmetic applicator for magnetic cosmetic products, comprising:
a housing defining a reservoir for containing a magnetic cosmetic product;
an applicator configured to extend into the reservoir, the applicator including a magnet; and
a wiper extending into the reservoir, the wiper having a bottom end with an opening through which the applicator extends, the wiper configured to remove excess product from the applicator as the applicator is withdrawn from the reservoir.

2. The cosmetic applicator of claim 1, wherein the applicator includes a cap and an elongate stem, at least a portion of the elongate stem configured to extend through the opening in the wiper and into the reservoir when the cap is attached to the housing.

3. The cosmetic applicator of claim 2, wherein the magnet is completely enclosed within the elongate stem.

4. The cosmetic applicator of claim 3, wherein a majority of the stem extends through the opening in the wiper and into the reservoir when the applicator is attached to the housing.

5. The cosmetic applicator of claim 3, wherein a majority of the stem is disposed within the wiper above the opening when the applicator is attached to the housing.

6. The cosmetic applicator of claim 2, wherein the magnet is disposed within a recess in an end of the elongate stem.

7. The cosmetic applicator of claim 6, wherein at least a portion of the magnet is in direct contact with cosmetic product when the stem is disposed within the reservoir.

8. The cosmetic applicator of claim 7, wherein a majority of the stem extends through the opening in the wiper and into the reservoir when the applicator is attached to the housing.

9. The cosmetic applicator of claim 7, wherein an entirety of the magnet is disposed within the reservoir when the applicator is attached to the housing.

10. The cosmetic applicator of claim 3, wherein a majority of the stem is disposed within the wiper above the opening when the applicator is attached to the housing.

11. The cosmetic applicator of claim 6, wherein the magnet is disposed with the recess such that only a single end surface of the magnet is exposed and in direct contact with cosmetic product when the stem is disposed within the reservoir.

12. The cosmetic applicator of claim 6, wherein the magnet is an elongate magnet extending from the end of the elongate stem, wherein sides and an end surface of the magnet are exposed and in direct contact with cosmetic product when the stem is disposed within the reservoir.

13. The cosmetic applicator of claim 12, wherein only the magnet extends through the opening in the wiper and into the reservoir when the applicator is connected to the housing.

14. The cosmetic applicator of claim 1, further comprising a collar attached to an upper open end of the housing, the collar connecting the applicator to the housing.

15. The cosmetic applicator of claim 1, wherein the bottom end of the wiper is disposed adjacent a bottom end of the reservoir.

16. The cosmetic applicator of claim 1, wherein the housing has a neck region defining an open end of the reservoir, wherein the bottom end of the wiper is disposed with the neck region of the housing.

17. The cosmetic applicator of claim 1, wherein the applicator takes the form of a mascara brush.

18. The cosmetic applicator of claim 1, wherein the applicator takes the form of a sponge.

19. The cosmetic applicator of claim 1, further comprising a magnetic cosmetic product contained in the reservoir.

20. A cosmetic applicator for magnetic cosmetic products, comprising:
a housing;
an applicator stem attached to the housing, the applicator stem including a magnet; and
a cap removably connected to the applicator stem, the cap covering an entirety of the applicator stem.
